(19) United States
(12) Patent Application Publication
(10) Pub. No.: US 2024/0102898 A1
(43) Pub. Date: Mar. 28, 2024

(54) IMPROVED METHOD FOR OPTICALLY CLEARING A TISSUE SAMPLE FOR A LIGHT MICROSCOPY EXAMINATION

(71) Applicant: MobiCron GmbH, Göttingen (DE)

(72) Inventors: Dr. Stephan Diekmann, Diessen (DE); Dr. Fred S. Wouters-Bunt, Göttingen (DE)

(21) Appl. No.: 18/257,565

(22) PCT Filed: Dec. 15, 2021
(86) PCT No.: PCT/EP2021/085981

§ 371 Date: Jun. 14, 2023

(30) Foreign Application Priority Data
Dec. 17, 2020 (EP) 20215089.2

(51) Int. Cl. G01N 1/30; G01N 33/483

(52) U.S. Cl. CPC G01N 1/30; G01N 33/4833

(57) ABSTRACT

The invention relates to a method for producing transparent tissue samples of a biological or human tissue for a light microscopy examination, having the steps a) dewatering the tissue sample using a dewatering solvent and b) clearing the dewatered tissue sample by transferring same into an embedding medium which contains an aromatic ester. The aromatic ester is a phenylacetic acid ester. In comparison to the benzyl benzoate/benzyl alcohol mixtures and methyl salicylate-benzyl benzoate mixtures conventionally used until now as embedding media, the aromatic ester has the advantage of being usable as a pure substance, in exactly the same way as a dibenzyl ether: Because the pure substance already has the desired refractive index, the refractive index does not need to be set by mixing the embedding medium. Furthermore, the aromatic ester is non-toxic, has a low degree of volatility, and allows a cold storage of the transparent tissue.

### RELATED APPLICATION INFORMATION

This patent claims priority from International PCT Patent Application No. PCT/EP2021/085981, filed Dec. 15, 2021 entitled, “IMPROVED METHOD FOR OPTICALLY CLEARING A TISSUE SAMPLE FOR A LIGHT MICROSCOPY EXAMINATION”, which claims priority to European Patent Application No. 20215089.2, filed Dec. 17, 2020, all of which are incorporated herein by reference in their entirety.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

The invention relates to a method according to the preamble of claim 1, to a kit for preparing biological tissue samples as claimed in claim 10, and to a use as claimed in claim 13.

Transparent biological tissue samples are needed in order for tissue samples to be able to undergo three-dimensional imaging, for example by light-sheet microscopy. The achievement of transparency in biological preparations necessitates the removal from the biological preparations of heme groups of the blood pigment hemoglobin and lipids in particular. In the “dehydrating” processes, the tissue is treated with various mixtures of a water-miscible organic solvent and water. The treatment is performed with an increasing proportion of the organic solvent in order to completely remove the water from the tissue. Here there are a number of options, for example tetrahydrofuran, methanol, isopropanol, tert-butanol, and ethanol. for tissue clearing in pathology ethanol is currently the most commonly used dehydration medium. The end result of all “dehydrating” processes is an anhydrous sample.

The final step in the various clearing methods is the adjustment of the refractive index to the refractive index of the tissue undergoing microscopy. The refractive index of dehydrated tissue is according to Spalteholz (“Uber das Durchsichtigmachen von menschlichen and tierischen Praparaten” [On the transparency of human and animal preparations], published by S. Hirzel, 1911, German Reich Patent No. 229044) n=1.547 for bone. The refractive index of other tissues can be estimated at approx. n=1.551 based on the optimal mixtures that he determined empirically. Such a high refractive index requires the use of aromatic compounds, which are generally immiscible or only slightly miscible with water. For his investigations, Spalteholz used mixtures of wintergreen oil (methyl salicylate) and benzyl benzoate or isosafrole in a mixing ratio tailored to the various tissues.

A variant of the Spalteholz method is known from Dodt, Leischner, Schierloh, Jahrling, Mauch, Deininger, Deussing, Eder, Zieglgansberger, and Becker “Ultramicroscopy: three-dimensional visualization of neuronal networks in the whole mouse brain”. Nat Methods. 2007; 4(4): 331-6.), in which “Murray's clear” (a 2:1 mixture of benzyl benzoate and benzyl alcohol with a refractive index of n=1.559) is used as the embedding medium. Another variant is known from Becker, Jahrling, Saghafi, Weiler, and Dodt H “Chemical Clearing and Dehydration of GFP Expressing Mouse Brains” (2012) PLoS ONE 7(3): e33916. https://doi.org/10.1371/journal.pone.0033916. Spalteholz embedding media and a number of other aromatic compounds are tested. Dibenzyl ether (DBE, refractive index n=1.562) emerged as the most suitable compound and has become the de facto gold standard in tissue clearing. The advantage of DBE is also that it is easy to use because the solution does not need to be mixed and its refractive index then checked; it can be used directly in the form of the pure substance.

WO 2017/093323 A1 discloses the use of ethyl cinnamate (ECi) and related cinnamyl esters as non-toxic alternatives to the embedding media in common use up till then. It exhibits lower toxicity than the other aromatic compounds used for tissue treatment, such as dibenzyl ether, benzyl alcohol or benzyl benzoate. Since the desired refractive index is achieved by the pure substance, this eliminates a mixing step and thus a work step that is a possible source of error.

The selection of pure liquid substances that are suitable as embedding media for dehydrated samples is thus currently limited to the two compounds dibenzyl ether and ethyl cinnamate. However, both substances have drawbacks: dibenzyl ether is toxic. Although ethyl cinnamate has low toxicity, its melting point is 6-9° C., so samples cleared with ethyl cinnamate cannot be stored in a refrigerator because the embedding medium will crystallize there. Ethyl cinnamate also has a comparatively high vapor pressure.

The object of the invention is to provide a method for preparing a transparent tissue sample of a biological tissue for examination by light microscopy that overcomes the above disadvantages and that in particular involves less effort than having to produce mixtures as the embedding medium, avoids embedding media with high toxicity, and permits the refrigerated storage of the dehydrated tissue samples.

The object is achieved according to the invention by a method as claimed in claim 1. The object is additionally achieved by a kit as claimed in claim 10 and by the use as claimed in claim 13.

Further embodiments are the subject matter of the dependent claims or described below.

The method according to the invention for preparing transparent tissue samples of a biological tissue for examination by light microscopy comprises the steps of:
a) dehydrating the tissue sample with a dehydrating solvent and
b) clearing the dehydrated tissue sample by placing it in a liquid embedding medium having a refractive index that matches the tissue.

The embedding medium comprises an aromatic ester corresponding to formula I or formula IV:

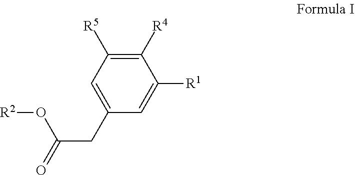

where
R¹ is —H, —CH₃ or —CH₂—CH₃,
R² is a residue of the formula II or formula III

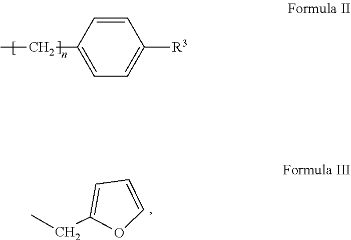

R³ is —H, —CH₃, —CH₂—CH₃ or —OCH₃,
R⁴ is —H or —CH₃,
R⁵ is —H or —CH₃, and
n is 1 or 2;
or

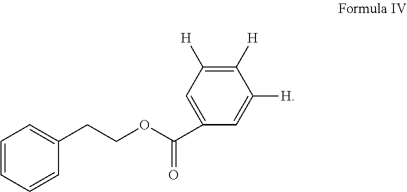

The ester according to the invention is preferably an aromatic ester corresponding to formula I.

The purpose of the method according to the invention is to achieve optical transparency in a biological or human tissue sample for light microscopy. The biological tissue is for example human tissue or animal tissue.

The clearing step of the clearing method is the adjustment of the refractive index to the refractive index of the tissue undergoing microscopy. For this, the tissue sample is transferred to a solution for adjustment of the refractive index: the embedding medium. The embedding medium must be miscible with the solvent used in the dehydration step.

In one embodiment, the embedding medium contains 10% to 100% by volume of the aromatic ester and 0% to 90% by volume of an optically suitable, inert organic solvent having a refractive index of about 1.3, preferably about 1.5. For example, the embedding medium contains 90% to 100% by volume of the aromatic ester corresponding to formula I.

In another embodiment, the embedding medium contains 10% to 100% by volume of the aromatic ester and 0% to 90% by volume of an optically suitable, inert organic solvent having a refractive index of about 2.0, preferably about 1.65. For example, the embedding medium contains 90% to 100% by volume of the aromatic ester corresponding to formula I.

All percentages according to the invention are percentages by volume (vol %).

In the preferred embodiment, the embedding medium consists of an aromatic ester corresponding to formula I or to formula IV, preferably to formula I, i.e. the aromatic ester is used as the pure substance. The pure substance is here understood as meaning the ester in the technically available purity.

The aromatic ester is preferably an ester of phenylacetic acid with aromatic alcohols. The aromatic ester is more preferably selected from benzyl 2-phenylacetate, 4-methoxybenzyl phenylacetate, phenethyl benzoate or 2-furylmethyl phenylacetate, particularly preferably benzyl 2-phenylacetate. Benzyl 2-phenylacetate (CAS No. 102-16-9) as the technical product has a purity of approx. 98.0%. 4-Methoxybenzyl phenylacetate (CAS No. 102-17-0) as the technical product has a purity of approx. 99.0%. 2-Phenethyl phenylacetate (CAS No. 102-20-5) as the technical product has a purity of approx. 99.0%. 2-Furylmethyl phenylacetate (CAS No. 36707-28-5) as the technical product has a purity of approx. 96.0%. Phenethyl benzoate (CAS No. 94-47-3) as the technical product has a purity of approx. 98%.

The purpose of the dehydration step a) is to obtain a water-free tissue sample. To completely remove the water from the tissue, the tissue sample is in a preferred embodiment preferably treated in a number of passages with various dehydrating compositions in a decreasing water series, i.e. mixtures containing an increasing proportion of a water-miscible organic solvent. Alcohols, ethers or ketones are used as the dehydrating medium. Suitable dehydrating solvents are for example ethanol, methanol, isopropanol, tert-butanol (IUPAC: 2-methylpropan-2-ol), tetrahydrofuran or acetone. In the method according to the invention, the dehydration step a) involves for example the use of dehydrating compositions.
composed of aqueous ethanol having increasing concentrations of ethanol, wherein the ethanol concentrations of the dehydrating compositions range from 30% to 100% by volume, or
composed of aqueous tetrahydrofuran having increasing concentrations of tetrahydrofuran, wherein the tetrahydrofuran concentrations of the dehydrating compositions range from 30% to 100% by volume, or
composed of aqueous methanol having increasing concentrations of methanol, wherein the methanol concentrations of the dehydrating compositions range from 30% to 100% by volume, or
composed of an aqueous mixture of another alcohol, ether or ketone, wherein the solvent concentration of the dehydrating compositions ranges from 30% to 100% by volume.

The dehydration medium here has the following properties: 1. it is completely miscible with water in order that the water and the fixative can be gradually removed from the tissue by means of an increasing concentration series, and 2. it is completely miscible with the embedding medium with which the refractive index is adjusted to that of the dehydrated tissue.

In an alternative embodiment, the dehydration step a) is carried out with 2,2-dimethoxypropane (DMP), which removes said water from the tissue by a chemical reaction with the water present in the tissue.

In one embodiment of the method according to the invention, the tissue sample, before it is dehydrated in step a) and optically cleared in step b), is
fixed and/or
fixed with formaldehyde and/or
washed and/or
washed with water and/or
incubated in an aqueous alkaline solution containing nonionic detergent and/or
delipidated with a detergent solution (delipidation) and/or
delipidated with an organic solvent (delipidation) and/or
bleached with oxidizing reagents and/or
decolorized with amino alcohols.

In one embodiment of the method according to the invention, the tissue sample, before it is dehydrated in step a) and optically cleared in step b), is fixed and the fixing agent is selected from
crosslinking fixatives such as formaldehyde, glutaraldehyde, acrolein, carbodiimides, diethyl pyrocarbonate, bisimidoesters or glyoxal or mixtures thereof, and/or
coagulant fixatives such as alcohols and other organic solvents, acids, potassium dichromate, lead nitrate, copper sulfate, and mercuric chloride and mixtures thereof.

The method according to the invention is preferably performed after tissue preparation steps and after electrophoresis steps. The tissue sample treated in the method according to the invention will thus preferably already have been pretreated. The pretreatment by electrophoresis is preferably carried out according to the electrophoretic clearing method described in DE 10 2016 123 458 B3. For the performance of the electrophoretic clearing, reference is made to patent specification DE 10 2016 123 458 B3, the content of which is hereby incorporated into this application.

In the method according to the invention, the optically cleared tissue sample is preferably in a further step examined under a microscope in order to obtain an image of the internal structure of the sample, the microscope being a light microscope, preferably a light-sheet microscope, a confocal microscope, a two-photon microscope or an optical projection tomography (OPT) microscope.

The embedding medium dibenzyl ether (DBE) used up to now has a refractive index of n=1.562. The second pure substance ethyl cinnamate has a refractive index of n=1.559.

Of the aromatic esters corresponding to formula I, phenylmethyl benzylacetate has for example a refractive index of n=1.555-1.558, 4-methoxybenzyl phenylacetate a refractive index of n=1.559, 2-phenethyl phenylacetate a refractive index of n=1.551, and 2-furylmethyl phenylacetate a refractive index of n=1.548 (calculated). Use of the aromatic esters corresponding to formula I as an embedding medium for tissue clearing of dehydrated tissue samples achieved a transparency identical to that of tissue samples treated with methyl salicylate/benzyl benzoate and with ethyl cinnamate. Not only alkoxy substituents, but also in particular short alkyl substituents on the phenylacetic esters give rise to refractive indices in this range, which is similar to the refractive indices of tissues determined by Spalteholz.

The studies by Spalteholz had previously suggested that the refractive indices of different tissues differ slightly from one other. In his original 1911 publication a series of human tissues are listed in order of increasing refractive index determined empirically based on the optimal mixing ratio of methyl salicylate and BB: young embryos (5:1-3:1 depending on weight)<adult decalcified bones (5:3) n=1.547<adult muscle approximately the same as older embryos (2:1)<brain and spinal cord (1:1). This variability has direct consequences for the transparency of different tissues and for the examination of said tissues by microscopy, and for quantitative spectroscopic determinations that can be performed with a microscope in particular. The method according to the invention offers the advantage that various pure substances are used as the embedding medium, which can be selected according to tissue type so as to approximate to the refractive index of the tissue.

Compared to the mixtures typically used as embedding media up to now benzyl benzoate/benzyl alcohol mixtures (BABB) and methyl salicylate/benzyl benzoate, the aromatic esters corresponding to formula I have the advantage that, just like dibenzyl ether, they can be used in the form of the pure substance: Since the desired refractive index is already achieved by the pure substance, there is no need for the refractive index to be set through the mixing of the embedding medium. This eliminates a mixing step and thus a superfluous work step and a possible source of error.

The aromatic esters corresponding to formula I also have low toxicity. This is advantageous when used as an embedding medium, since the tissue sample in the microscope is positioned immediately below the user's airways and the user can inhale the vapor rising from the sample. According to the European Chemicals Agency ECHA, the toxicity of phenylmethyl benzylacetate is not a toxicological concern, unlike that of dibenzyl ether or benzyl benzoate/benzyl alcohol mixtures.

In addition, the phenylacetic esters corresponding to formula I are of low volatility, since they have a significantly low vapor pressure than the known embedding media, which means that the tissue sample gives rise to less vapor. This additionally reduces the already low toxicity of the aromatic esters corresponding to formula I.

A disadvantage of the aromatic compounds used up to now as embedding media is their corrosive effect. The aromatic compounds are known to attack the lens and the microscope itself and just the vapor from these volatile compounds is known to be enough over time to corrode the microscope's liquid-facing surfaces. The lower volatility of the aromatic esters corresponding to formula I means that the embedding media according to the invention offer significantly higher material compatibility, since they give rise to appreciably less vapor that could lead to corrosion. In summary, the low volatility of the aromatic esters corresponding to formula I reduces odor and respiratory irritation/toxicity, and also the corrosive effect of the vapor on the microscope.

Phenylmethyl benzylacetate has moreover a pleasant sweet and floral odor, with notes of jasmine and chocolate, which increases acceptance of the embedding medium by laboratory workers. Thus, during microscopy the user is not troubled by disagreeable odors rising from the stage, as is the case with the embedding media conventionally used: dibenzyl ether has a disagreeable plastic-like odor and benzyl alcohol a phenolic odor.

In addition, the phenylacetic esters corresponding to formula I and formula IV allow the cleared samples to be stored in a refrigerator. The melting point of phenylmethyl benzylacetate, for example, is between −13.54° C. and −9° C., i.e. the compound can be kept in a refrigerator and even permits storage or transport on ice or in cool packs.

The kit according to the invention for preparing biological tissue samples for light microscopy comprises:
a dehydrating solvent for dehydrating the tissue sample and
an embedding medium for clearing the dehydrated tissue sample by placing the sample in the embedding medium,

where

the embedding medium is an aromatic ester of the formula I or formula IV as described above. Preferably, the aromatic ester in the kit according to the invention is benzyl 2-phenylacetate, 4-methoxybenzyl phenylacetate, phenethyl benzoate or 2-furylmethyl phenylacetate, preferably benzyl 2-phenylacetate. The aromatic ester is preferably employed in the concentrations and purities described above.

The dehydrating solvent in the kit is preferably an ether, ketone or alcohol, preferably the solvent is selected from ethanol, methanol, isopropanol, tert-butanol, 2,2′-thiodiethanol, trichloroethanol, tetrahydrofuran or acetone.

According to the invention, an aromatic ester corresponding to formula I or formula IV, where

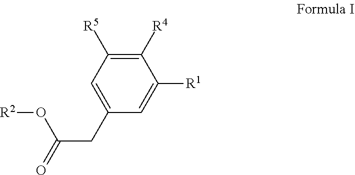

where
R¹ is —H, —CH₃ or —CH₂—CH₃,
R² is a residue of the formula II or formula III

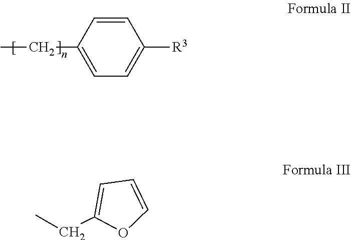

R³ is —H, —CH₃, —CH₂—CH₃ or —OCH₃,
R⁴ is —H or —CH₃,
R⁵ is —H or —CH₃, and
n is 1 or 2;
or

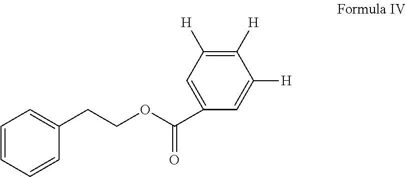 [SMILES: O=C(OCCc1ccccc1)c1ccccc1]

is used as an embedding medium for preparing a biological, in particular human, tissue sample for examination by light microscopy.

The aromatic ester is preferably benzyl 2-phenylacetate, 4-methoxybenzyl phenylacetate, phenethyl benzoate or 2-furylmethyl phenylacetate, more preferably benzyl 2-phenylacetate. The aromatic ester is preferably used in the concentrations and purities described above.

The vapor pressures of various embedding media are known from the literature.

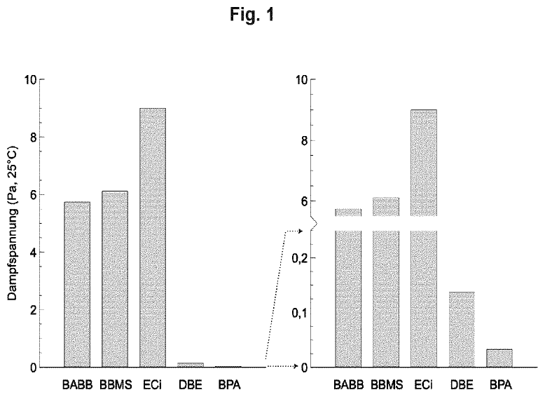

FIG. 1 shows the vapor pressures of various embedding media. The phenylmethyl benzylacetate (benzyl phenylacetate (BPA)) of the invention has a significantly lower vapor pressure than the embedding media used according to the current state of the art. BPA is the least volatile, with the lowest vapor pressure of the embedding media that were compared. The vapor pressure of BPA (at 25° C.) is approximately 275 times lower than that of ethyl cinnamate. The two embedding media BABB and BBMS likewise have significantly (about 180 times) higher vapor pressures.

In the expanded section in FIG. 1 it can be seen that BPA is even less volatile (about 4 times less volatile) than DBE, which likewise contains two aromatic ring groups and is in turn less volatile than ECi on account of its significantly lower vapor pressure, with a lower value than methyl salicylate (including in the mixture with BB) and ECi.

### EXAMPLES

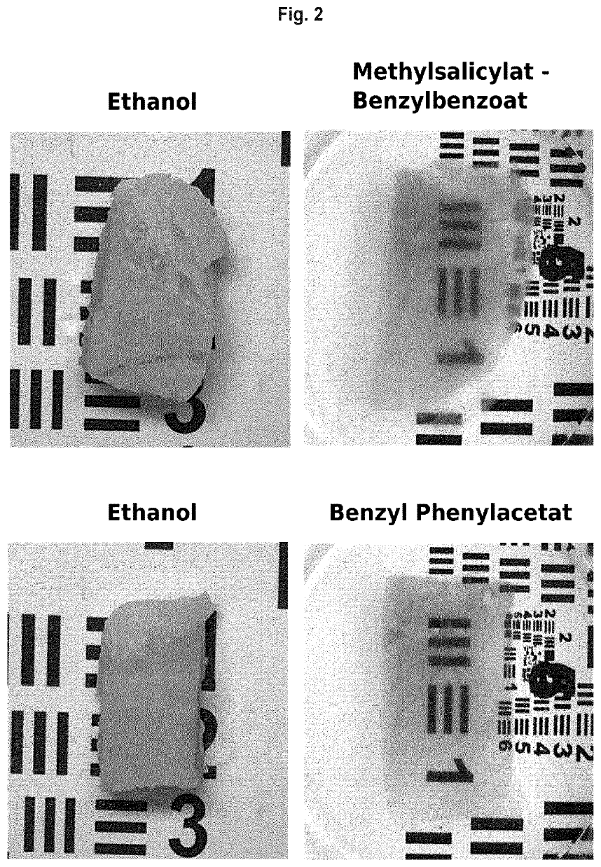

A tissue sample (pig lung) having a volume of about 0.25 ml (0.5×0.5×1 cm 3) was dehydrated with ethanol in several passages. In the final passage, high-purity ethanol was used and the dehydrated tissue sample was photographed. The results are shown in FIG. 2, left-hand side. The tissue sample was then placed in 5 ml of the respective embedding media for 3 hours. The embedding media used were a mixture of methyl salicylate with benzyl benzoate, and the benzyl phenylacetate of the invention. The result of the clearing step was also photographed and shown on the right-hand side of FIG. 2.

It was found that methyl salicylate/benzyl benzoate already results in good transparency in the tissue sample. However, the transparency achieved with the benzyl phenylacetate of the invention was even better still.

The invention is not restricted to one of the embodiments described above, but may be modified in a variety of ways.

All the features and advantages apparent from the claims, the description, and the drawing, including method steps, may be essential to the invention either on their own or in the diversity of combinations.

1. A method for preparing transparent tissue samples of a biological tissue for examination by light microscopy, the method comprising the steps of:
a) dehydrating the tissue sample with a dehydrating solvent and
b) clearing the dehydrated tissue sample by placing it in an embedding medium comprising an aromatic ester corresponding to formula I or formula IV, where 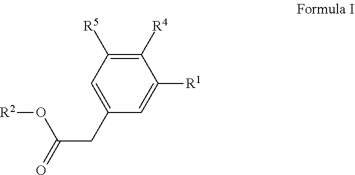
where
R¹ is —H, —CH₃ or —CH₂—CH₃,
R² is a residue of the formula II or formula III 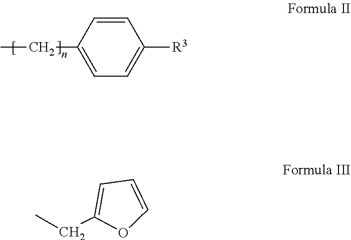
R³ is —H, —CH₃, —CH₂—CH₃ or —OCH₃,
R⁴ is —H or —CH₃,
R⁵ is —H or —CH₃, and
n is 1 or 2;
or

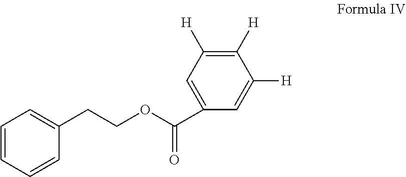 [SMILES: O=C(OCCc1ccccc1)c1ccccc1]

2. The method as claimed in claim 1, wherein the embedding medium contains 10% to 100% by volume of the aromatic ester and 0% to 90% by volume of an optically suitable, inert organic solvent having a refractive index of about 1.3, preferably 1.5.

3. The method as claimed in claim 1, wherein the embedding medium contains 10% to 100% by volume of the aromatic ester and 0% to 90% by volume of an optically suitable, inert organic solvent having a refractive index of about 2.0, preferably 1.65.

4. The method as claimed in claim 1, wherein the embedding medium consists of an aromatic ester corresponding to formula I or formula IV.

5. The method as claimed in claim 1, wherein the aromatic ester is benzyl 2-phenylacetate, 4-methoxybenzyl phenylacetate, phenethyl benzoate or 2-furylmethyl phenylacetate, preferably benzyl 2-phenylacetate.

6. The method as claimed in claim 1, wherein the dehydration step a) involves the use of dehydrating compositions composed of aqueous alcohol, ketone or ether.

7. The method as claimed in claim 1, wherein the tissue sample, before it is dehydrated in step a) and optically cleared in step b), at least one of:
is fixed
is fixed with formaldehyde
is washed
is washed with water
is incubated in an aqueous alkaline solution
is delipidated with a detergent solution
is delipidated with an organic solvent
is bleached with oxidizing reagents
is decolorized with amino alcohols.

8. The method as claimed in claim 1, wherein the tissue sample, before it is dehydrated in step a) and optically cleared in step b), is fixed and that the fixative is selected from crosslinking fixatives such as formaldehyde, glutaraldehyde, acrolein, carbodiimides, diethyl pyrocarbonate, bisimidoesters or glyoxal or mixtures thereof, and/or coagulant fixatives such as alcohols and other organic solvents, acids, potassium dichromate, lead nitrate, copper sulfate, and mercuric chloride and mixtures thereof.

9. The method as claimed in claim 1, wherein the optically cleared tissue sample is in a further step examined under a microscope in order to obtain an image of the internal structure of the sample, the microscope being a light microscope.

10. A kit for preparing biological tissue samples for light microscopy, the kit comprising:
a dehydrating solvent for dehydrating the tissue sample and
an embedding medium for clearing the dehydrated tissue sample by placing the sample in the embedding medium,
wherein the dehydrating solvent is an alcohol, ketone or ether and the embedding medium is an aromatic ester of the formula I or formula IV, where 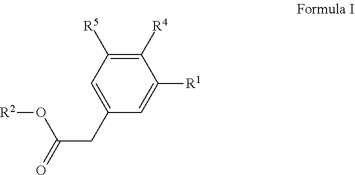
where
R¹ is —H, —CH₃ or —CH₂—CH₃,
R² is a residue of the formula II or formula III 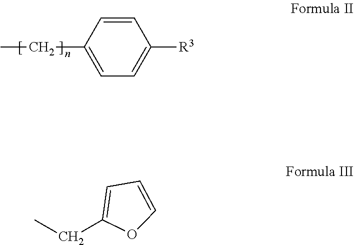
R³ is —H, —CH₃, —CH₂—CH₃ or —OCH₃,
R⁴ is —H or —CH₃,
R⁵ is —H or —CH₃, and
n is 1 or 2;
or

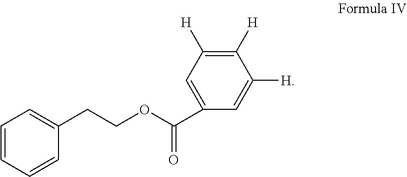

11. The kit as claimed in claim 10, wherein the aromatic ester is benzyl 2-phenylacetate, 4-methoxybenzyl phenylacetate, phenethyl benzoate or 2-furylmethyl phenylacetate, preferably benzyl 2-phenylacetate.

12. The kit as claimed in claim 10, wherein the dehydrating solvent is selected from ethanol, methanol, isopropanol, tert-butanol, 2,2′-thiodiethanol, trichloroethanol, tetrahydrofuran or acetone.

13. The method for use of an aromatic ester corresponding to formula I or corresponding to formula IV, where 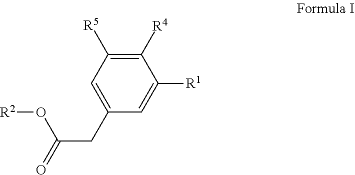
where
R¹ is —H, —CH₃ or —CH₂—CH₃,
R² is a residue of the formula II or formula III 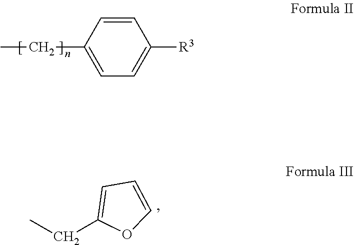
R³ is —H, —CH₃, —CH₂—CH₃ or —OCH₃,
R⁴ is —H or —CH₃,
R⁵ is —H or —CH₃, and
n is 1 or 2; or 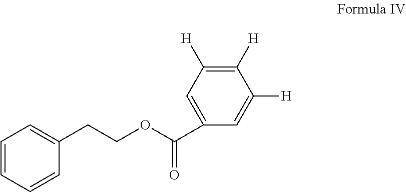 [SMILES: O=C(OCCc1ccccc1)c1ccccc1]
as an embedding medium for preparing a biological tissue sample for examination by light microscopy.
